(19) United States
(12) Patent Application Publication
(10) Pub. No.: US 2019/0183845 A1
(43) Pub. Date: Jun. 20, 2019

(54) Cream and Oil for Facial Rejuvenation

(71) Applicant: Dr. Magana Products, Inc., Temecula, CA (US)

(72) Inventors: Jose Luis Magaña Garcia, Puerto Vallarta (MX); Liliana Marisalto Ruiseco Lopez, Puerto Vallarta (MX)

(73) Assignee: Dr. Magana Products, Inc., Temecula, CA (US)

(21) Appl. No.: 16/221,167

(22) Filed: Dec. 14, 2018

Related U.S. Application Data
(60) Provisional application No. 62/607,248, filed on Dec. 18, 2017.

(51) Int. Cl. A61K 31/352; A61K 36/58; A61K 36/889; A61K 8/97

(52) U.S. Cl. CPC A61K 31/352; A61K 8/97; A61K 36/889; A61K 36/58

(57) ABSTRACT

A mixture and method of production of a topical formulation for facial rejuvenation using cannabis extract that results in the remedy of alterations in a skin's appearance when applied to such skin.

This patent application claims the benefit of U.S. Provisional Patent Application Ser. No. 62/607,248, entitled “Cream and Oil for Facial Rejuvenation,” filed Dec. 18, 2017, which is incorporated in its entirety here by this reference.

### TECHNICAL FIELD

This invention relates to a composition for cream and oil for facial rejuvenation and its method of production.

### BACKGROUND

Throughout time, there have been few solutions that truly address the issue of skin blemishes of all kinds. A person's appearance is incredibly important to their confidence in life and the present invention seeks to help those who suffer from issues related to skin problems. Tins invention is a solution to that problem that overcomes skin based blemishes of all kinds through a new mixture that is disclosed below.

### SUMMARY

The present invention is directed to a topical formulation for skin care, which in one embodiment may come in the form of creams and oils for facial rejuvenation. This invention also covers the method of production that, in one embodiment, may result in a cream that when applied to skin remedies any alteration in the skin's appearance. In one embodiment, the composition may comprises a mixture for facial rejuvenation comprising: (a) approximately 200 to 400 ml of coconut oil; (b) approximately 100 to 300 ml of Moringa oleifera; (c) approximately 200 to 400 ml of avocado oil; (d) approximately 30 ml of lavender oil; (e) approximately 50 to 150 ml of a marijuana extract; (f) approximately 50 to 150 ml of a neem extract; (g) wherein the mixture remedies any alteration in a person's skin appearance when applied.

These and other features, aspects, and advantages of the present invention will become better understood with reference to the following description and appended claims.

### DETAILED DESCRIPTION OF THE INVENTION

The detailed description set forth below is intended as a description of presently-preferred embodiments of the invention and is not intended to represent the only forms in which the present invention may be constructed or utilized. The description sets forth the functions and the sequence of steps for constructing and operating the invention in connection with the illustrated embodiments. It is to be understood, however, that the same or equivalent functions and sequences may be accomplished by different embodiments that are also intended to be encompassed within the spirit and scope of the invention.

Description of Procedure for the Elaboration of Ollilis Dermal Local Nutrient Cream

Stage I. Obtaining Extracts

I. 1.0—Marijuana (Cannabis sativa) Extract

The process begins by weighing out an amount of Cannabis sativa. For example, in one embodiment, 50 to 150 g of Cannabis sativa may be used. In one embodiment, preference is given to the buds or inflorescences from female cannabis plants, than to other parts of the plant that contain more THC (tetrahydrocannabinol). Before weighing the plant, twigs and other elements of the plant are eliminated until only the Cannabis buds remain.

I. 1.2—Next, in one embodiment a container may be used to simmer coconut oil for approximately 24 hours. In one embodiment approximately 500 to 1,500 ml of coconut oil (Cocos nucifera) may be simmered in a double boiler at a low temperature, for example less than 100° C., taking care that the solution does not exceed a maximum of approximately 70° C., that way the plant will be macerated, in order to facilitate that both the phytonutrients and the nutraccuticals substances “dissolve” in the oil.

Then, the cannabis/oil suspension may be completely liquefied. In one embodiment, the liquefaction of the cannabis/oil may utilize a speed blender in order to obtain, at most, an homogeneous plant/oil mixture. Next, the “maceration” process continues for an additional period of time. For example, the additional maceration of the plant in the oil may be for another approximately 24 hours. This additional maceration step may be accomplished by using, for example, a double boiler at a maximum temperature of 70° C., in order extract the most of the active substances from the cannabis plant and to ensure that during the filtering, there will be only a minimum part of the phytonutrients/nutraceuticals left in tire bagasse.

I. 1.3—Next, the solution may be filtered to separate cannabis bagasse from cannabis phytonutrient and cannabis nutraceutical saturated oils. For this purpose, in one embodiment, a press/filter may be used, which may be designed and built ad hoc with stainless steel and topped with an approximately 5 micron mesh (Widman International). Subsequently the oil/phytonutrients-nutraceuticals solution may be passed through an additional filter. In one embodiment a paper filter, such as Whatman grade 1, may be used for this additional filtering step. This additional filtering step is taken to obtain a completely transparent “solution” of oil with no solid particles suspended.

I. 2.0—Neem (Asadirachta indica) Extract

I. 2.1—The next step begins by thoroughly cleaning and weighing out dried neem leaves. In one embodiment, for example, approximately 50 to 150 g of dried neem leaves may be cleaned and weighed out.

I. 2.2—Next, by using a container, place the leaves with coconut oil (Coco nucifera) to begin to macerate the leaves for a period of time. For example, in one embodiment, approximately 500 to 1,000 ml of coconut oil (Coco nucifera) may be used to macerate the leaves. In one embodiment, for example, the maceration process lasts for up to a maximum of approximately 24 hours at up to a maximum temperature of 70° C. During the maceration process care may be taken to reduce the time of blending so the solution will be slightly liquefied, this is due to the feet that the neem leaves may be easily pulverized resulting in a consistency that will cause difficulties during the process of filtering.

I. 2.3—Next, the process comprises filtering the solution to separate neem bagasse from neem phytonutrient and neem nutraceutical saturated oils. In one embodiment, for example, a press/filter may be used, which may be designed and built ad hoc with stainless steel and topped with an approximately 5 micron mesh (Widman International). Subsequently, the oil/phytonutrients-nutraceuticals solution is passed through a filter. In one embodiment, this additional filtering step may utilize a paper filter such as Whatman grade 1 paper filters. This additional filtering step is conducted to obtain a completely transparent “solution” of oil, with no solid particles suspended.

Stage II. Elaboration of the Mixture: Dermal Local Nutrient Cream

II. 1.1.—The next step in the process it to place in a container coconut oil with beeswax. For example, in one embodiment a Pyrex glass container may be used to place approximately 100 to 300 ml of coconut oil and approximately 25 to 100 g of finely grated beeswax. Next, the coconut oil and beeswax mixture is simmered. In one embodiment, the simmering is conducted in a double boiler at approximately 70° C. Next, the mixture is stirred constantly so the wax may melt gradually until a homogeneous mixture forms. Next, more coconut oil is added to the mixture. For example, in one embodiment, approximately 100 to 300 ml of coconut oil is used to create a uniform mixture of beeswax and coconut oil.

II. 1.2.—Next, the stirring continues consistently and avocado oil is added to the mixture. For example, in one embodiment, while stirring constantly, approximately 300 to 500 ml of avocado oil may be used, previously simmered in, for example, a double boiler at a maximum temperature of approximately 70° C., in order to not modify the solution.

II. 1.3.—Still stirring constantly, to the mixture of coconut and avocado oils, the cannabis extract that was made in step 1.0 above is added to the mixture. In one embodiment, for example, approximately 50 to 150 ml of cannabis extract may be used.

II. 1.4.—Next, without stopping the constant stirring, the neem extract that was made in step 2.0 from above is added to the mixture. In one embodiment, for example, approximately 50 to 150 ml of the neem extract is used.

II. 1.5.—Next, and while constantly stirring, lavender oil (Aura cacia) is added to the mixture. In one embodiment, approximately 5 to 15 ml of lavender oil may be used. This step completes the preparation of coconut oil, beeswax, avocado oil, the extracts of cannabis, and neem that will yield the final mixture that we call DERMAL LOCAL NUTRIENT CREAM, conceived and designed to be applied locally on the skin, to remedy practically any alteration in its appearance.

II. 1.6.—The final step is to fill containers with the mixture. In one embodiment, this is done while maintaining the mixture at a constant temperature of approximately 70° C., arid stirring continuously while 30 ml (2 fl. Oz) of the mixture is poured into, for example approximately 38 ml capacity glass jars

This Combination of Oils and Extracts Guarantees a Local Dermal Contribution of Phytonutrients:

The following list of vitamins and minerals may be used to contribute to the phytonutrients of the present invention. The combination of vitamins may include, for example, carotene, thiamine, riboflavin, niacin, pantothenic acid, pyridoxine, ascorbic acid, vitamin E. The combination of minerals may include, for example, calcium, iron, magnesium, phosphorus, potassium, zinc. Additional ingredients may be included in one embodiment such as lauric acid, cryptoxanthin, lutein+zeaxanthin, nimbidin, nimbolide, mahmoodin.

Stage III. Elaboration of the Mixture of the Nutrient Cream and Oil for Facial Rejuvenation

III. 1—To begin the third stage of the process avocado oil is placed in a container, which is in turn placed in a water bath. In one embodiment, a Pyrex glass container may be used to place, for example, approximately 200 to 400 ml of avocado oil. Next, in one embodiment, the container may be placed in water bath at maximum temperature of approximately 70° C. The process continues while the oil is constantly stirred and then coconut oil and moringa oil are added to the mixture. In one embodiment, for example, approximately 200 to 300 ml of coconut oil may be added and then also, approximately 100 to 300 ml of moringa oil may be added. Additionally, in an embodiment, this current mixture may be heated to approximately 70° C.

The next step in the process is to add the cannabis extract and the neem extract that was made in previous steps. In one embodiment, for example, approximately 50 to 150 ml of cannabis extract and approximately 75 to 300 ml of neem extract may be added to the mixture. Finally, lavender oil may be added to the mixture. In one embodiment, up to approximately 30 ml of essential lavender oil may be added while stirring constantly.

Each of the above referenced combinations may be generally referred to as mixtures.

Nutrient Cream for Facial Rejuvenation

In an additional embodiment similar steps as above are utilized. However, in an additional embodiment, beeswax from above may be replaced by Moringa Oil. This additional embodiment may use, for example, a large container of approximately 2000 mL capacity.

Next, in one embodiment, a water bath may be used at, for example, a maximum temperature of approximately 70° C. In one embodiment, for example, the process may add the following: (1) Coconut oil (approximately 200 to 400 mL is poured; it is stirred and brought to the same temperature); (2) Avacado oil (approximately 200 to 400 mL is poured; it is stirred and brought to the same temperature); (3) Moringa oleifera (approximately 100 to 300 mL is poured; it is stirred and brought to the same temperature); (4) Marijuana extract (approximately 50 to 150 mL); (5) Neem extract (approximately 50 to 150 mL); and (6) Lavander (up to approximately 30 mL).

After this process the product is ready to be poured in the glass containers.

What is claimed is:

1. A method for producing topical formulation for skin care, comprising:
(a) a first step for producing a cannabis solution comprising:
(1) eliminating twigs from a cannabis plant;
(2) weighing the cannabis plant;
(3) combining the cannabis plant with a first volume of coconut oil to form the cannabis solution;
(4) macerating the cannabis solution for approximately 24 hours;
(5) liquefying the cannabis solution;
(6) macerating the cannabis solution for at least another 24 hours;
(7) filtering the cannabis solution, wherein the filtering separates a cannabis bagasse from a cannabis phytonutrient and a cannabis nutraceutical saturated oil from the cannabis solution;
(8) passing the cannabis solution through a first paper filter;
(9) whereby a transparent solution of cannabis extract is formed;
(b) a second step conducted after the first step for producing a neem solution comprising:
(1) cleaning dried neem leaves;
(2) weighing dried neem leaves;
(3) combining the dried neem leaves with a second volume of coconut oil to form the neem solution;
(4) macerating the neem solution for up to 24 hours;
(5) blending the neem solution;
(6) filtering the neem solution, wherein the filtering process separates a neem bagasse from a neem phytonutrient and a neem nutraceutical saturated oil from the solution;
(8) passing the neem solution through a second paper filter;
(9) whereby a transparent solution of neem extract is formed;
(c) a third step conducted after the first and the second step for elaboration of a mixture comprising:
(1) mixing a third coconut oil with finely grated beeswax to form a first mixture;
(2) simmering the first mixture;
(3) adding a fourth coconut oil to the first mixture to form a second mixture;
(4) adding avocado oil to the second mixture to form a third mixture;
(5) combining the third mixture with the transparent solution of cannabis extract to form a fourth mixture;
(6) combining the fourth mixture with the transparent solution of neem extract to form a fifth mixture;
(7) combining the fifth mixture with lavender oil to form a topical formulation;
(8) whereby when the topical formulation is applied to skin, the topical formulation remedies alterations in the skin's appearance.

2. The method as in claim 1, wherein filtration of the cannabis solution uses a stainless steel container with approximately 5 micron mesh.

3. The method as in claim 2, wherein filtration of the neem solution uses a stainless steel container with approximately 5 micron mesh

4. The method as in claim 3, wherein the cannabis is extracted from a female marijuana plant,

5. The method as in claim 4, wherein the simmering has a maximum temperature of 70 degrees Celsius.

6. The method as in claim wherein a double boiler is used for the simmering,

7. The method as in claim 6, wherein the topical formulation is placed into 38 ml capacity glass jars.

8. The method as in claim 7, wherein the beeswax is replaced with Moringa oleifera,

9. A mixture for facial rejuvenation comprising:
(a) a coconut oil;
(b) an organic material;
(c) an avocado oil;
(d) a lavender oil;
(e) a marijuana extract comprising;
(1) a cannabis plant; and
(2) a coconut oil;
(f) a neem extract comprising;
(1) dried neem leaves; and
(2) a coconut oil;
(g) wherein the mixture remedies alterations in a person's skin when applied.

10. The mixture as in claim 9, wherein the organic material is Moringa oleifera.

11. The mixture as in claim 9, wherein the organic material is beeswax.

12. The mixture as in claim 9, wherein the coconut oil is unrefined coconut oil.

13. The mixture as in claim 9, wherein the coconut oil is refined coconut oil.

14. A method for producing topical formulation for skin care, comprising:
(a) producing a cannabis solution comprising:
(1) eliminating twigs from a cannabis plant;
(2) weighing the cannabis plant;
(3) combining the cannabis plant with a first volume of coconut oil to form the cannabis solution;
(4) macerating the cannabis solution for approximately 24 hours;
(5) liquefying the cannabis solution;
(6) macerating the cannabis solution for at least another 24 hours;
(7) filtering the cannabis solution, wherein the filtering separates a cannabis bagasse from a cannabis phytonutrient and a cannabis nutraceutical saturated oil from the cannabis solution;
(8) passing the cannabis solution through a first paper filter;
(9) whereby a transparent solution of cannabis extract is formed;
(b) producing a neem solution comprising:
(1) cleaning dried neem leaves;
(2) weighing dried neem leaves;
(3) combining the dried neem leaves with a second volume of coconut oil to form the neem solution;
(4) macerating the neem solution for up to 24 hours;
(5) blending the neem solution;
(6) filtering the neem solution, wherein the filtering process separates a neem bagasse from a neem phytonutrient and a neem nutraceutical saturated oil from the solution;
(8) passing the neem solution through a second paper filter;
(9) whereby a transparent solution of neem extract is formed;
(c) elaboration of a mixture comprising:
(1) mixing a third coconut oil with finely grated beeswax to form a first mixture;
(2) simmering the first mixture at a maximum temperature of 70 degrees Celsius;
(3) adding a fourth coconut oil to the first mixture to form a second mixture;
(4) adding avocado oil to the second mixture to form a third mixture;
(5) combining the third mixture with the transparent solution of cannabis extract to form a fourth mixture;
(6) combining the fourth mixture with the transparent solution of neem extract to form a fifth mixture;
(7) combining the fifth mixture with lavender oil to form a topical formulation;
(8) whereby when the topical formulation is applied to skin, the topical formulation remedies alterations in the skin's appearance.

15. The method as in claim 14, wherein filtration of the cannabis solution uses a stainless steel container with approximately 5 micron mesh.

16. The method as in claim 14, wherein filtration of the neem solution uses a stainless steel container with approximately 5 micron mesh.

17. The method as in claim 14, wherein the cannabis is extracted from a female marijuana plant.

18. The method as in claim 14, wherein the simmering has a maximum temperature of 70 degrees Celsius.

19. The method as in claim 14, wherein a double boiler is used for the simmering.

20. The method as in claim 14, wherein the beeswax is replaced with Moringa oleifera.
